(19) United States
(12) Patent Application Publication
(10) Pub. No.: US 2022/0068477 A1
(43) Pub. Date: Mar. 3, 2022

(54) ADAPTABLE REINFORCEMENT LEARNING

(71) Applicant: International Business Machines Corporation, Armonk, NY (US)

(72) Inventors: VENITA GLASFURD, Bangalore (IN); ANJALI SHAH, Short Hills, NJ (US); Daniella DSouza, Grapevine, TX (US)

(21) Appl. No.: 17/008,702

(22) Filed: Sep. 1, 2020

(51) Int. Cl. G16H 50/20; G06K 9/62; G06N 20/00

(52) U.S. Cl. CPC G16H 50/20; G06N 20/00; G06K 9/6267

(57) ABSTRACT

Embodiments of the present invention provide a computer system, a computer program product, and a method that comprises collecting an input from a user by transmitting instructions to at least one sensor device in a plurality of senor devices; dynamically classifying a volatile chemical by analyzing at least one result within a plurality of results for a chemical identification based on a collected input; determining a status of the user based on an analysis of an environment of the user and a dynamic classification of the volatile chemical; and generating an adaptative model that assesses a determined status, the dynamic classification of the volatile chemical, and the collected input into a user interface within a computing device.

### BACKGROUND

The present invention relates generally to the field of healthcare, and specifically reinforcement learning within the healthcare field.

Machine learning is the study of computer algorithms that improve automatically through experience. It is seen as a subset of artificial intelligence. Machine learning algorithms build a mathematical model based on sample data, known as training data, in order to make predictions or decisions without being explicitly programmed to do so. Machine learning algorithms are used in a wide variety of applications where it is difficult or infeasible to develop conventional algorithms to perform the needed tasks. Machine learning is closely related to computational statistics, which focuses on making predictions using computers.

Artificial intelligence, sometimes called machine intelligence, is intelligence demonstrated by machines, unlike the natural intelligence displayed by humans and animals. Modern machine capabilities generally classified as artificial intelligence include successfully understanding human speech, competing at the highest level in strategic game systems, autonomously operating cars, intelligent routing in content delivery networks, and military simulations. Artificial intelligence techniques have experienced a resurgence following concurrent advances in computer power, large amounts of data, and theoretical understanding; and artificial intelligence techniques have become an essential part of the technology industry, helping to solve many challenging problems in computer science, software engineering and operations research.

Reinforcement learning is an area of machine learning concerned with how software agents out to take action in an environment in order to maximize the notion of cumulative reward. Reinforcement learning is one of the three basic machine learning paradigms, alongside supervised learning and unsupervised learning. Reinforcement learning differs from supervised learning in not needing labelled input/output pairs be presented, and in not needing sub-optimal actions to be explicitly corrected. Instead the focus is on finding a balance between exploration (of uncharted territory) and exploitation (of current knowledge).

### SUMMARY

Embodiments of the present invention provide a computer system, a computer program product, and a method that comprises collecting an input from a user by transmitting instructions to at least one sensor device in a plurality of senor devices; dynamically classifying a volatile chemical by analyzing at least one result within a plurality of results for a chemical identification based on a collected input; determining a status of the user based on an analysis of an environment of the user and a dynamic classification of the volatile chemical; and generating an adaptative model that assesses a determined status, the dynamic classification of the volatile chemical, and the collected input into a user interface within a computing device.

### BRIEF DESCRIPTION OF THE DRAWINGS

FIG. 1 is a functional block diagram depicting an environment with a computing device connected to or in communication with another computing device, in accordance with at least one embodiment of the present invention;

FIG. 2 is a flowchart illustrating operational steps for generating an adaptive model based on comparison of monitored data to a generated baseline, in accordance with at least one embodiment of the present invention;

FIG. 3 is a flowchart 300 illustrating operational steps for generating an adaptive model that compares monitored data of a user to a generated baseline, in accordance with at least one embodiment of the present invention;

FIG. 4 is a flowchart illustrating operational steps for automatically updating the generated adaptive model to assess a condition of a user, in accordance with at least one embodiment of the present invention; and

FIG. 5 depicts a block diagram of components of computing systems within a computing display environment of FIG. 1, in accordance with an embodiment of the present invention.

### DETAILED DESCRIPTION

Embodiments of the present invention recognize the need for an improvement to current healthcare systems as acute shortages of home health aides and nursing assistants are increasing in developed countries and threatening care for people with serious disabilities and vulnerable older adults by implementing reinforcement learning algorithms to make long-term care of the elderly sustainable in the future. Embodiments of the present invention provide systems, methods, and computer program products for an improvement to existing adaptative healthcare technologies. Currently, learning agents and reinforced learning algorithms within healthcare technologies generated specifically for individuals that suffer from respiratory conditions, pulmonary conditions, chronic illness and other physical ailments. Embodiments of the present invention improve these current learning agents and reinforced learning algorithms by generating an adaptive model specifically for individuals that suffer from ailments that can affect a user's emotional nature in addition to generating an adaptive model for individuals that suffer from physical ailments. Emotional nature is defined as a conscious and subjective mental reaction toward a particular event and is usually accompanied by changes in the physiologic and behavioral aspects of a person that are detectable by sensor devices over a predetermined period of time. For example, embodiments of the present invention provide an improvement to learning agents and reinforced learning algorithms that assist in detecting aliments of an emotional nature for persons of all ages, the youth and the elderly. However, embodiments of the present invention provide an improvement to current learning agents and reinforced learning algorithms using adaptive indicators to continually detect emotional health of individuals by measuring volatile observed chemicals, user tone data, and user-specific geo-spatial data, and predict an efficient response to the detection of an ailment. Embodiments of the present invention generates an adaptive model detailing monitored ailments of a user by receiving input from a user over time, generating a baseline based on the received input for the user, monitoring data of the user in real-time using Internet of Things (“IoT”), generating an adaptative model that compares the monitored data to the generated baseline, and generating a notification to a computing device alerting the generation of the adaptative model to increase the efficiency in responding to ailments of a user.

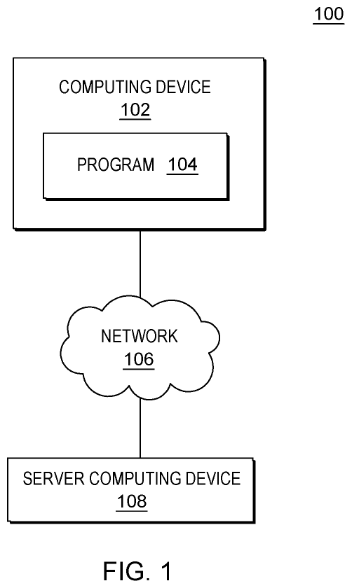

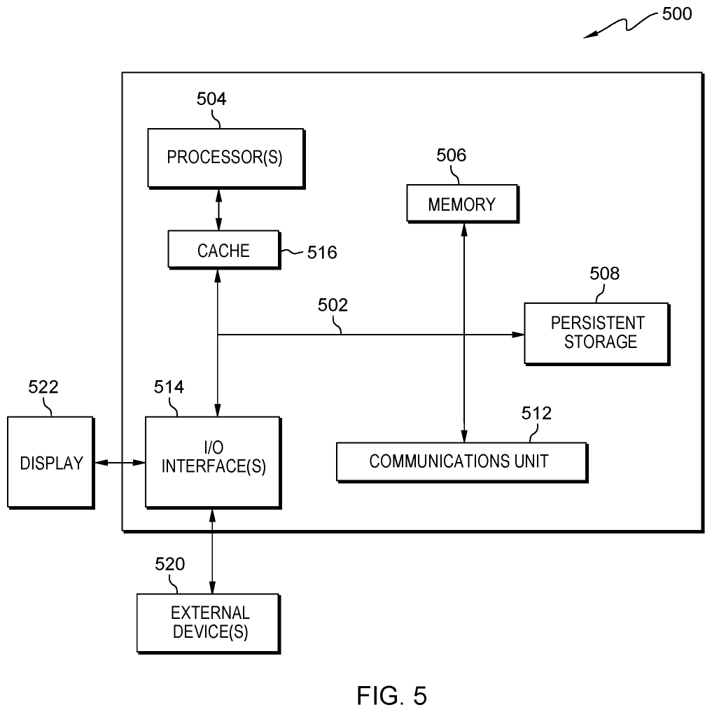

FIG. 1 is a functional block diagram of a computing environment 100 in accordance with an embodiment of the present invention. The computing environment 100 includes a computing device 102 and a server computing device 108. The computing device 102 and the server computing device 108 may be desktop computers, laptop computers, specialized computer servers, smart phones, or any other computing devices known in the art. In certain embodiments, the computing device 102 and the server computing device 108 may represent computing devices utilizing multiple computers or components to act as a single pool of seamless resources when accessed through a network 106. The computing device 102 may include a sensing device or a plurality of sensing devices that include air indicators to observe and identify volatile chemicals in the air. The nature of the sensing device is based on the type of data observed and identified. Generally, the computing device 102 and the server computing device 108 may be representative of any electronic devices, or a combination of electronic devices, capable of executing machine-readable program instructions, as described in greater detail with regard to FIG. 5.

The computing device 102 may include a program 104. The program 104 may be a stand-alone program on the computing device 102. In another embodiment, the program 104 may be stored on a server computing device 108. In this embodiment, the program 104 triggers a reinforcement learning algorithm to support and help a user that is in distress, and the feedback to the reinforcement learning algorithm is defined as the response to that distress. For example, the program 104 triggers the reinforcement learning algorithm to support a prediction or trend in user action. In this embodiment, user action is defined as response to an event. For example, a user that is undergoing an event that entails emotional decline or a person who is recovering from an emotional traumatic situation. In this embodiment, the program 104 detects the emotional distress of the user in real-time using IoT data and machine learning by learning tone expressions of the user and predicting an ailment associated with the detected emotional distress and learned tone expressions. In this embodiment, the program 104 learns tone expressions by observing a verbal expression or a tone using sensor devices, storing the observed verbal expression or tone within a generated database, and analyzing the observed verbal expression or tone using a learning agent algorithm. In this embodiment, the program 104 assesses conditions associated with a user's mental health condition by identifying the analysis of the observed verbal expression or tone, comparing the identified analysis of the observed verbal expression or tone to a historical baseline of learned verbal expression or tones, and verifying the assessment of conditions by matching the identified analysis of the observed verbal expression to the comparison of the baseline of learned verbal expressions or tones. For example, the program 104 observes user A's speech pattern that predicts distress or sadness, whereas user B's speech pattern predicts happiness or calmness. In this embodiment, the program 104 dynamically generates an adaptive model using activity analysis, reinforcement learning algorithms, and classification analysis based on real-time IoT monitoring data in addition to a historical baseline of the user. For example, the program 104 monitors that a user is often assisted entering the bathroom and appears to be in need of assistance to go to the bathroom. In this embodiment, the program 104 dynamically triggers a reinforcement learning algorithm to help a user based on the generated baseline, or predetermined assistance parameters, and this assistance may or may not involve another user. For example, the program 104 detects a need involving an additional user's assistance by transmitting a generated notification to an operator. In this embodiment, the program 104 automatically collects feedback for the reinforcement learning algorithm by using IoT based monitoring. For example, the program 104 monitors user A at location A is in need of assistance, generates an adaptive model that details the rationale for the assistance, generates a notification based on the generated adaptive model, and transmits the generated notification to user B at location B.

In this embodiment, the program 104 receives input from a user; collects received input from the user over time; generates a baseline based on the received input for the user; monitors data of the user in real-time using Internet of Things (“IoT”); generates an adaptative model that compares the monitored data to the generated baseline; and generates a notification to a computing device alerting the generation of the adaptative model.

The network 106 can be a local area network (“LAN”), a wide area network (“WAN”) such as the Internet, or a combination of the two; and it may include wired, wireless or fiber optic connections. Generally, the network 106 can be any combination of connections and protocols that will support communication between the computing device 102 and the server computing device 108, specifically the program 104 in accordance with a desired embodiment of the invention.

The server computing device 108 may include the program 104 and may communicate with the computing device 102 via the network 106.

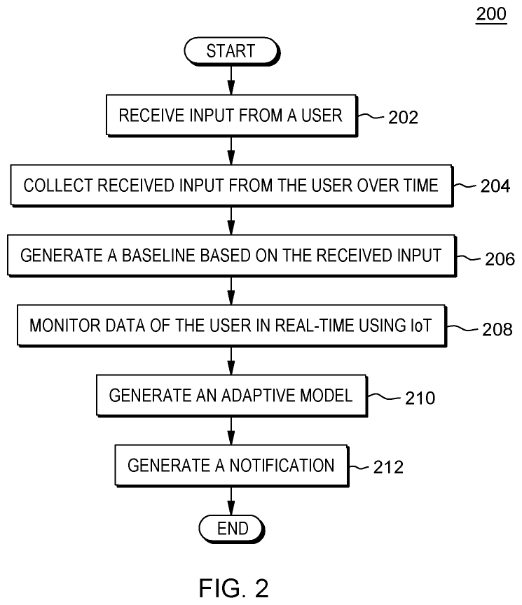

FIG. 2 is a flowchart 200 illustrating operational steps for generating an adaptive model based on comparison of monitored data to a generated baseline, in accordance with at least one embodiment of the present invention.

In step 202, the program 104 receives input from a user. In this embodiment, the program 104 receives input by accessing a database or a device associated with collecting data for at least one user. In this embodiment, the program 104 accesses the database at predetermined intervals or fixed intervals and transmits queries on demand to the database. In this embodiment, input is defined as data associated with a user that is collected. In another embodiment, input may include other information that is relevant to learning In this embodiment, the program 104 receives opt-in/opt-out permission from a user to gain access to input data associated with the user, and this permission allows the program 104 to receive input for the user. For example, the program receives biological data such as age, height, and weight for a user.

In step 204, the program 104 collects received input from the user over time. In this embodiment, the program 104 collects received input from the user by continually receiving input from the user and storing the received input in a database for future use. In this embodiment, collected input is defined as information related to the user. In another embodiment, collected input may be defined other relevant information associated with the user. For example, the program 104 continually collects data for the user for three months.

In step 206, the program 104 generates a baseline based on the received input for the user. In this embodiment, the program 104 generates a baseline based on the received and collected input by identifying similarities and differences in the received and collected input for a user. In this embodiment, the program 104 generates the baseline for the user to establish a historical context to detect changes to the baseline in the future. For example, the program 104 generates a baseline of historical biological data for the user that includes height, weight, age, blood pressure, and blood sugar baselines.

In step 208, the program 104 monitors data of the user in real-time using IoT. In this embodiment, the program 104 monitors health data associated with the user that would be received and collected in real-time using IoT systems to detect data such as air indicator data, emotion context data, and tone analysis data. For example, the program 104 actively monitors the user's breathing rates, heart rates, and levels of serotonin using IoT systems.

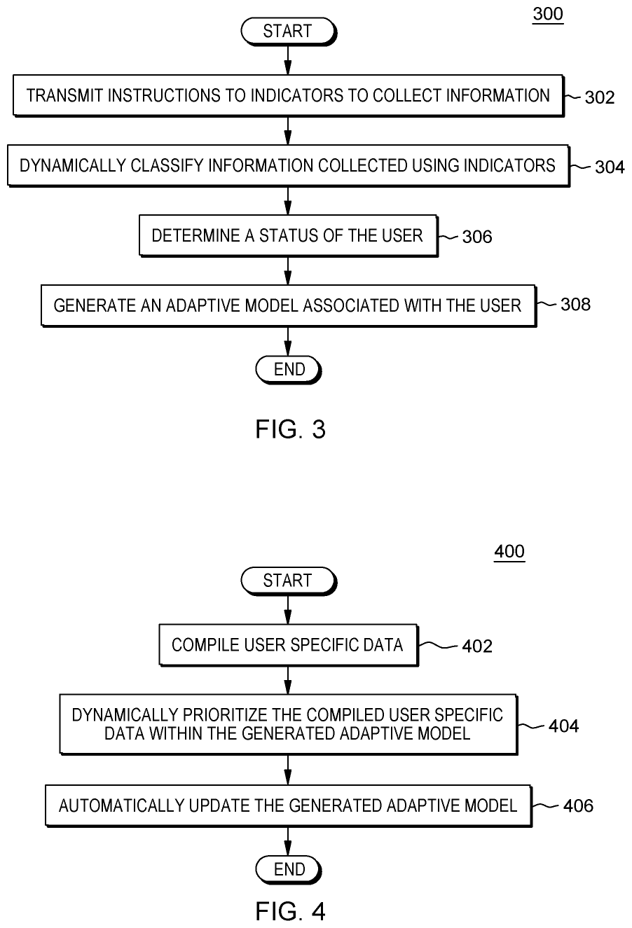

In step 210, the program 104 generates an adaptative model that compares the monitored data to the generated baseline. In this embodiment, the program 104 generates an adaptive model by compiling the monitored data, the generated baseline data, and ailment predictions associated with volatile observed chemicals and displaying the compilation of the data in a user interface within the computing device. In this embodiment, the program 104 used within the adaptive model is capable of comparing real-time monitored data to the generated baseline by using machine learning algorithms and reinforcement learning algorithms to dynamically detect outliers or anomalies in the compilation of data, such as an sudden observation of a specific volatile chemical that is associated with a new physiological ailment that is accompanied with a physical event. This step will be further explained in FIG. 3.

In this embodiment, the program 104 uses machine learning algorithms and reinforcement learning algorithms to detect volatile chemicals in the air by tracking the identity of the observed chemicals and their estimated density using sensor devices in the computing device 102, such as air indicators. In this embodiment, volatile chemicals are defined as volatolome and assist the program 104 in making critical or critical emotional diagnosis of emotional decline or trauma. In another embodiment, volatile chemicals may be defined as any type of chemical that may cause harm to the user or is a product of a change in the monitored input from the generated baseline for the user. For example, the program 104 generates a model that automatically changes in the user's emotional nature when the change is accompanied with a change in tone, and the generated adaptive model predicts more accurately the pourability of the user going into a state of emotional decline. For example, the program 104 assesses a change in the user's mental health by identifying a change in user's tone, a detected increase in breathing rate, an elevated heart rate, and minimal brain activity; comparing the identified changes to the historical baseline of data associated with the user, determining that the identified changes meets or exceeds a predetermined threshold based on the comparison to the baseline data, and verifying that the identified changes are associated with the ailment by matching the identified changes to conditions associated with the ailment.

In another embodiment, the program 104 assesses a change in the user's emotion. In this embodiment, the program 104 assesses a change in the user's emotion by establishing a baseline of emotional data associated with the user by continually collecting data associated with the user, identifying a deviation in the data by determining that a point of the collected data meets or exceeds a predetermined threshold of emotion, and verifying the identified deviation by determining the difference between the identified deviation and the established baseline for collected data. In this embodiment, the program 104 defines the established baseline as a user that is happy. Therefore, deviations that meet or exceed the predetermined threshold of emotion are defined as changes in emotion. For example, the program 104 establishes a baseline heart rate of 80 and a baseline tone volume of 60 decibels for the user, identifies a deviation by determining that an observed heart rate of 190 and an observed tone volume of 110 decibels, and verifies that the deviations meets or exceeds the predetermined threshold of emotion due to the increase in both heart rate and tone volume. This verification indicates that the user's emotional natures has changed.

In step 212, the program 104 generates a notification to a computing device 102. In this embodiment, the program 104 generates a notification to the computing device 102 to alert an operator of the computing device 102 of an identified outlier within the generated adaptative model. In this embodiment, the generated notification is used to assist an operator of the computing device 102 in treating the user, assisting the user, or transporting the user based on the adaptive model associated with the user. In this embodiment, the program 104 generates the notification to assess the ailment of the user based on the generated adaptive model. In this embodiment, the program 104 generates the notification and transmits the notification to the computing device 102 to determine the presence of the ailments of the user. In this embodiment, the program 104 generates the notification and transmits the notification by communicating with the computing device 102 using IoT systems. In another embodiment, the program 104 displays the generated adaptive model within a user interface, and this display serves as a generated notification.

FIG. 3 is a flowchart 300 illustrating operational steps for generating an adaptive model that compares monitored data of a user to a generated baseline, in accordance with at least one embodiment of the present invention.

In step 302, the program 104 transmits instructions to indicators located within a computing device 102 to collect information. In this embodiment, the program 104 activates the indicators located within the computing device 102 to collect and monitor factors of the location of the user. In this embodiment, the program 104 transmits instructions to the indicators of the computing device 102 to collect and monitor volatile chemicals emitted by humans. In another embodiment, the program 104 transmits instructions to IoT devices to collect and monitor factors of the user's location and status of the user.

In step 304, the program 104 dynamically classifies information collected using indicators. In this embodiment, the program 104 dynamically classifies volatile chemicals by collecting a sample of the volatile observed chemical by using an apparatus to trap an emitted chemical within a predetermined area. For example, the program 104 collects a sample using a breath analyzer.

In this embodiment and in response to collecting the sample of the volatile observed chemical, the program 104 compares the collected sample to a stored database of known sample by identifying the collected sample within the stored database of known samples using a machine learning algorithm. In this embodiment, the stored database of known samples is a pre-stored database supplied by the manufacturer. In this embodiment, the program 104 extracts information associated with the identified collected sample by matching the identified collected sample to a corresponding link within the stored database for identified collected sample. In this embodiment, the program 104 identifies any known information associated with the identified collected sample by examining the corresponding link within the database. In this embodiment, any known information also includes ailments associated with the presence of the volatile observed chemicals. For example, the program 104 identifies the presence of benzene within the breath analyzer, gathers all known information on benzene stored within the database, and compares the identified benzene with the information known on benzene.

In this embodiment and in response to comparing the collected sample to the database of known chemicals, the program 104 determines threshold percentage of the volatile observed chemical within the user's environment. In this embodiment, the program determines threshold percentage by calculating a chemical density of the volatile observed chemical and determining a threshold percentage of the volatile observed chemical by comparing the calculated chemical density to an estimated chemical density based on the known information from the database associated with the volatile observed chemical. In this embodiment, the threshold percentage is defined as the percentage of volume that the volatile observed chemical comprises of the current user's environment. In this embodiment, the program 104 uses the threshold percentage to obtain more information on the user's environment and identification of the volatile observed chemical because the threshold percentage may act as an identification marker, (i.e. chemical fingerprint) that establishes the presence of a volatile observed chemical versus an outlier or detected anomaly. For example, the program 104 determines the benzene meets threshold percentage by calculating the chemical density of the collected benzene and comparing that calculated density to the estimated chemical density of benzene stored in the database to further identify the volatile observed chemical of benzene is present in the user's environment.

In this embodiment and in response to determining the threshold percentage of the volatile observed chemical within the user's environment, the program 104 verifies the presence of the volatile observed chemical. In this embodiment, the program 104 verifies the presence of the volatile observed chemical at the calculated chemical density by placing the collected sample in a micro mass spectrometer to perform an analysis on the collected sample and verify the preceding steps of the program 104. In this embodiment, the program 104 classifies volatile observed chemicals by verifying the calculated chemical densities of the collected samples against the known chemicals stored within the database. For example, the program 104 verifies that the collected sample was benzene by dynamically placing the collected sample into a micro mass spectrometer to identify the elements, quantify the density of each element present, and verify a composition or calculated chemical density of the benzene.

In another embodiment, the program 104 dynamically performs a query of the pre-stored database by selecting a corresponding link associated with the collected sample and retrieving information from the selected corresponding link that provides additional detail on the collected sample. In this embodiment, the program 104 dynamically performs a query when comparing the collected sample to the database of known samples. In another embodiment, the program 104 dynamically performs a query of an external database, such as the internet, to provide additional information associated with the collected sample.

In another embodiment and in response to the program 104 failing to have any information associated with the collected sample located within the known sample database, the program 104 places the collected sample within the micro mass spectrometer to learn chemical density, weight, abundance, and any additional identifying factors associated with the collected sample and stores the learned information within the database of known samples for future use.

In another embodiment and in response to verifying the predetermined threshold of the collected sample, the program 104 classifies a species of volatile observed chemicals by determining the collected sample is a composition of multiple volatile observed chemicals, identifying the composition to the corresponding link within the known database, retrieving any species information associated with the composition within the known database, and verifying the calculated chemical density of the composition in relation to the estimated chemical density and known chemical density retrieved within the known database. In this embodiment, the program 104 has a manufacturer provided database that stores multiple observations of volatile chemicals and ailments associated with the observed volatile chemicals. In this embodiment and in response to classifying the species of volatile chemicals, the program 104 dynamically classifies volatile chemicals by examining the performed query and the identified similarities and differences and comparing the identified similarities of the classified species of observed volatile chemicals using the learning agent algorithms.

In another embodiment, the program 104 dynamically identifies a type of volatile chemical emitted and the amount or quantified volume of volatile chemicals emitted from a user using a learning agent algorithm, which examines the identified volatile chemical, counts a number of identified volatile chemicals, and then identifies a species of a volatile chemicals by comparing the counted volatile chemicals within a predetermined similarity threshold that indicates the counted volatile chemicals is the identified volatile chemical, to count the number volatile chemicals observed and to identify the species of volatile chemicals observed by the program 104 through the indicators.

In step 306, the program 104 determines a status of the user. In this embodiment, the program 104 determines the status of the user by analyzing the dynamically classified information by examining the classified information, extracting commonalities within the examined classified information, and predicting an ailment by performing an assessment of the extracted commonalties of the examined classification information and using the reinforced learning algorithm for continually observing behavior of the user; and transmitting observed behavior and analyzed information to a user interface of a computing device 102. For example, the program 104 determines the status of the user by analyzing volatile chemicals emitted in that air traditionally emitted with chronic heart failure and predicts a heart attack, observes tightness or pain in the chest, back, and neck, of the user as well as fatigue and lightheadedness, and transmits these findings to the smart tablet so an operator can assess whether the user is suffering from a heart attack based on the classified information.

In this embodiment, the program 104 determines a status of the user by exploring the user's environment and exploiting a response to generated alerts for volatile observed chemicals to predict an assessment of the user using the learning agent algorithm. In this embodiment and in response to exploring and exploiting, the program 104 determines a status of the user by training the reinforcement learning algorithm to provide scalar feedback as the learning agent learns to sense and assess the user's environment via generated adaptive models and alerts. In this embodiment, the program 104 trains the reinforcement learning algorithm by creating an environment around the user, assigning a positive value to a predetermined action or progression within a user's physiological ailment, assigning a negative value to a predetermined action or progression within a user's physiological ailment, calculating an overall score associated with the user's physiological ailment, and dynamically modifying the calculated overall score based on newly received information associated with the user's environment. In this embodiment and in response to training the reinforcement learning algorithm, the program 104 calculates as overall score for the assessment of the user by assigning values to factors of the determination of the status based on the training performed by the reinforcement learning algorithm. In this embodiment, the factors are defined as contextual factors and assigned quantitive values proportional to their impact. In this embodiment, the program 104 recalculates the overall score based on newly received information, where predictions that are determined as correct based on the analysis of the learning agent results in a positive value and predictions that are determined to be incorrect based on the analysis of the learning agent results in a negative value. In this embodiment, a positive value increases the overall score, and a negative value deceases the overall score. In this embodiment, the program 104 calculates an overall score by aggregating the assigned values.

In another embodiment, the program 104 determines a status of the user; analyzes the dynamically classified information by assessing the user's ailment using a classification algorithm, which predicts an ailment by leveraging the extracted commonalties of the classified information; determining all possible ailments associated with the extracted commonalities; removing any ailment that does not reach a predetermined threshold of extracted commonalities; and providing all remaining ailments associated with the volatile chemicals emitted by the user. In this embodiment, the classification algorithm classifies the user's ailment, generates an alert based on the classified ailment, and predicts progression of the user's ailments based on the training of the program 104 and comparing the volatile observed chemicals and their estimated density to the historical data. In this embodiment, the program 104 determines the status of the user to assist in the care-giving or assessment of the user's ailments through an dynamic alert generation using the reinforcement learning algorithm.

In another embodiment and in response to analyzing the dynamically classified information, the program 104 transmits instructions to sensors in the IoT devices and the computing device 102 to continually observe behavior of the user by detecting movement and other factors of the user, such as speech patterns for tone analysis, using a machine learning algorithm. In this embodiment, the program 104 learns behavior by storing the continually observed behavior, examining the continually observed behavior, and determining a trend within the examined continually observed behavior using machine learning algorithms. In another embodiment, the program 104 has pre-saved learned behaviors that are used to compare unknown observed behavior to learned behaviors of the user for future comparison by extracting commonalties using the machine learning algorithm. In this embodiment, the program 104 creates an environment around the user using IoT device in conjunction with multiple sensor devices.

In another embodiment and in response to continually observing behavior of the user, the program 104 transmits observed behavior and analyzed information by communicating with the computing device 102. In this embodiment, the program 104 defines status as a condition, behavior, or mood of the user. For example, the program 104 observes multiple statuses for a single user, such as sleepy, in distress, not breathing, hungry, and thirsty, etc. In this embodiment, the program 104 determines the status of the user using the classification algorithm to observe an action or the classified information, where the action or classified information is affected by the environment of the user.

In this embodiment and in response to observing an action or classified information, the program 104 transmits the observed action and classified information to the computing device 102 and dynamically receives feedback from an operator, where the operator is a trained professional that can understand when the observed action or classified information determines that a user is in distress. In this embodiment and in response to receiving feedback from the operator, the program 104 stores the received feedback and observed actions within the computing device 102. For example, the program 104 observes a change in tone or emitted volatile observed chemicals accompanied with a spike in heart rate and decrease in blood pressure in a user; transmits the increased heart rate and decreased blood pressure to the smart device associated with the user; the nurse, or operator, recommends a sedative to assist the user; and the program 104 stores the information of the assistance, such as time, date, and reason for the sedative. In another embodiment, the program 104 automatically injects the sedative into the user without the need of the operator being within a predetermined proximity of the user.

In step 308, the program 104 generates an adaptative model associated with the user. In this embodiment, the program 104 generates the adaptive model by compiling the determined status, the predicted ailment, and classification of the volatile chemicals emitted by the user; prioritizing the determined status, the predicted ailment, and classification of the volatile chemicals using the machine learning algorithm to rank and sort the received input; and automatically updating the generated adaptive model using the reinforcement learning algorithm in a database that is displayed via a user interface in the computing device 102. This step is further explained in FIG. 4.

In another embodiment, the program 104 updates the adaptive model in response to any new collected information, observed action, received feedback, and changes in determined status at fixed predetermined intervals. In this embodiment, the program 104 generates the adaptive model to detail the collected information, observed actions, and received feedback of the user, and the program 104 transmits the generated adaptive model to other computing devices 102 to improve in the efficiency of assessing a different user. In another embodiment, the program 104 generates the adaptive model based on information and recommendations that the operator of the program 104 manually inputs for the user.

FIG. 4 is a flowchart 400 illustrating operational steps for assessing a condition of a user by automatically updating the generated adaptive model, in accordance with at least one embodiment of the present invention.

In step 402, the program 104 compiles user specific data. In this embodiment, the program 104 compiles user specific data by identifying the user specific data and transmitting that data to a database. In this embodiment, the program 104 stores the identified user specific data on the database and saves the identified user specific data on the database. In another embodiment, the program 104 stores the identified user specific data on the server computing device 108 via the network 106.

In other embodiments, the program 104 compiles user specific data by identifying the determined status, the predicted ailment, and the classification of emitted volatile chemicals using the learning algorithm. User specific data is defined as data used to assist an operator in assessing an ailment of the user. For example, user specific data includes the determined status, the predicted ailment, and the classification of emitted volatile chemicals.

In this embodiment, the program 104 compiles user specific data in the generated adaptive model to display the user specific data in a user interface of the computing device 102. In another embodiment, the program 104 compiles the user specific data within a server computing device 108. In this embodiment, the program 104 complies the user specific data within a server computing device 108 via the network 106. For example, the program 104 compiles the thirsty status, the classification for lack of detected insulin chemicals, the prediction of diabetes, and alert generation for appropriate care assessment in the generated adaptive model within the application in the smart tablet.

In step 404, the program 104 dynamically prioritizes the compiled user specific data within the generated adaptive model. In this embodiment, the program 104 dynamically prioritizes the compiled user specific data by assigning values to multiple factors of the user specific data, calculating an overall score for each form of user specific data, and arranging the overall score for each form of user specific data in a sequential manner having overall scores having a greater value assigned a higher order than overall scores having a lesser value using machine learning algorithms within the generated adaptive model displayed in the user interface of the computing device 102. The overall score is defined as the summation of the assigned values of the multiple factors of each form of the user specific data. For example, the program 104 assigns multiple values for the determined status, the predicted ailment, and the observed volatile chemicals and adds those assigned values to calculate the overall score.

In this embodiment, the program 104 dynamically prioritizes the compiled user specific data by ranking the compiled user data based on the overall score and sorting the compiled user data in sequential order based on arranged overall scores. For example, the program 104 prioritizes the classification of lack of detected insulin chemicals at a higher order than the determined thirsty status due to the classification of volatile chemicals having a greater overall score than the determined status.

In step 406, the program 104 automatically updates the generated adaptive model. In this embodiment, the program 104 automatically updates the generated adaptive model by analyzing the prioritized order of the user specific input by examining the dynamically calculated overall scores and verifying the prioritized order of the user specific input based on extracted commonalities within the user specific input; identifying changes in the generated adaptive model by receiving new user input, recalculating overall scores based on new received user input, and determining a difference between the recalculated overall score and the original overall score; and modifying the generated adaptive model to reflect the identified changes using the reinforced learning algorithm. For example, the program 104 recalculates an overall score for the determined status in light of newly received information that affects a factor, determines the differences between the recalculated overall score and the original overall score, and optimizes the generated adaptive model in response to determining the identified changes or determined differences between the recalculated overall score and the original overall score.

In another embodiment, the program 104 automatically updates the generated adaptive model using the reinforced learning algorithm without the need of manual input from an operator or user. In another embodiment, the program 104 updates the generated adaptive model using the reinforced learning algorithm on a fixed time interval. For example, the program 104 automatically updates the adaptative model in response to identifying a change in the determined status of the user. In another example, the program 104 updates the adaptive model every 30 seconds to reflect any identified changes.

FIG. 5 depicts a block diagram of components of computing systems within a computing environment 100 of FIG. 1, in accordance with an embodiment of the present invention. It should be appreciated that FIG. 5 provides only an illustration of one implementation and does not imply any limitations with regard to the environments in which different embodiments can be implemented. Many modifications to the depicted environment can be made.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

A computer system 500 includes a communications fabric 502, which provides communications between a cache 516, a memory 506, a persistent storage 508, a communications unit 512, and an input/output (I/O) interface(s) 514. The communications fabric 502 can be implemented with any architecture designed for passing data and/or control information between processors (such as microprocessors, communications and network processors, etc.), system memory, peripheral devices, and any other hardware components within a system. For example, the communications fabric 502 can be implemented with one or more buses or a crossbar switch.

The memory 506 and the persistent storage 508 are computer readable storage media. In this embodiment, the memory 506 includes random access memory (RAM). In general, the memory 506 can include any suitable volatile or non-volatile computer readable storage media. The cache 516 is a fast memory that enhances the performance of the computer processor(s) 504 by holding recently accessed data, and data near accessed data, from the memory 506.

The program 104 may be stored in the persistent storage 508 and in the memory 506 for execution by one or more of the respective computer processors 504 via the cache 516. In an embodiment, the persistent storage 508 includes a magnetic hard disk drive. Alternatively, or in addition to a magnetic hard disk drive, the persistent storage 508 can include a solid state hard drive, a semiconductor storage device, read-only memory (ROM), erasable programmable read-only memory (EPROM), flash memory, or any other computer readable storage media that is capable of storing program instructions or digital information.

The media used by the persistent storage 508 may also be removable. For example, a removable hard drive may be used for the persistent storage 508. Other examples include optical and magnetic disks, thumb drives, and smart cards that are inserted into a drive for transfer onto another computer readable storage medium that is also part of the persistent storage 508.

The communications unit 512, in these examples, provides for communications with other data processing systems or devices. In these examples, the communications unit 512 includes one or more network interface cards. The communications unit 512 may provide communications through the use of either or both physical and wireless communications links. The program 104 may be downloaded to the persistent storage 508 through the communications unit 512.

The I/O interface(s) 514 allows for input and output of data with other devices that may be connected to a mobile device, an approval device, and/or the server computing device 108. For example, the I/O interface 514 may provide a connection to external devices 520 such as a keyboard, keypad, a touch screen, and/or some other suitable input device. External devices 520 can also include portable computer readable storage media such as, for example, thumb drives, portable optical or magnetic disks, and memory cards. Software and data used to practice embodiments of the present invention, e.g., the program 104, can be stored on such portable computer readable storage media and can be loaded onto the persistent storage 508 via the I/O interface(s) 514. The I/O interface(s) 514 also connect to a display 522.

The display 522 provides a mechanism to display data to a user and may be, for example, a computer monitor.

The present invention may be a system, a method, and/or a computer program product. The computer program product may include a computer readable storage medium (or media) having computer readable program instructions thereon for causing a processor to carry out aspects of the present invention.

The computer readable storage medium can be any tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer implemented method comprising:
collecting an input from a user by transmitting instructions to at least one sensor device in a plurality of senor devices, wherein the input comprises information associated with the user;
dynamically classifying a volatile chemical by analyzing at least one result within a plurality of results for a chemical identification based on a collected input;
determining a status of the user based on an analysis of an environment of the user and a dynamic classification of the volatile chemical, wherein the status is a condition, mood, or emotion of the user; and
generating an adaptative model that assesses a determined status, the dynamic classification of the volatile chemical, and the collected input into a user interface within a computing device.

2. The computer-implemented method of claim 1, wherein dynamically classifying a volatile chemical by analyzing at least one result within a plurality of results for a chemical identification based on the collected input using multiple algorithms comprises:
determining that a collected input matches a respective known sample in a database of known samples;
calculating a chemical density for each collected input that matches the respective known sample based on chemical identification markers associated with a density of the respective known samples in the database of known samples;
determining a threshold percentage of the collected input within an environment of the user by comparing the calculated chemical density to an estimated chemical density of the respective known samples; and
verifying the determined threshold percentage of the identified collected input.

3. The computer-implemented method of claim 1, wherein determining a status of the user comprises:
examining the dynamically classified information;
extracting commonalities within the examined classified information;
predicting an ailment by performing an assessment of an extracted commonalty of the examined classification information associated with a plurality of contextual factors that indicate a presence of an ailment; and
continually observing behavior of the user by transmitting instructions to the at least one sensor device in a plurality of sensors devices.

4. The computer-implemented method of claim 1 further comprising automatically updating the generated adaptive model by:
transmitting data that is specific to the user to the generated adaptive model;
dynamically prioritizing each contextual factor within a compiled data based on the user specific data within the generated adaptive model; and
automatically updating the generated adaptive model based on a quantitive value of the contextual factors.

5. The computer-implemented method of claim 4, wherein dynamically prioritizing each contextual factor within a compiled data comprises:
assigning quantitative values to each contextual factor in a plurality of the contextual factors associated with the user specific data, wherein each contextual factor is associated with a respective user;
calculating an overall score respective of the contextual factors associated with the user specific data, wherein the overall score is a summation of the assigned quantitative values of the contextual factors; and
arranging the respective overall score of the contextual factors associated with the user specific data in a sequential manner having overall scores having a greater value assigned a higher order than the overall scores having a lesser value using machine learning algorithms.

6. The computer-implemented method of claim 4, wherein automatically updating the generated adaptive model based on a quantitive value of the contextual factors comprises:
verifying the prioritized order of the user specific input based on extracted commonalities within the user specific input;
identifying changes in the generated adaptive model by determining a difference between a recalculated overall score and an original calculated overall score; and
modifying the generated adaptive model to reflect the identified changes using a reinforced learning algorithm.

7. The computer-implemented method of claim 1, wherein determining a status for a user comprises:
creating an environment around the user using IoT devices in conjunction with sensor devices;
assigning a positive value to a predetermined action or progression within a user's physiological ailment and assigning a negative value to a predetermined action or progression within a user's physiological ailment;
calculating an overall score associated with the user's physiological ailment by aggregating the assigned values of the predetermined actions or progressions within the user's physiological ailment; and
in response to receiving additional information associated with a user's environment and physiological ailment, dynamically modifying the calculated overall score.

8. The computer-implemented method of claim 1 further comprising assessing a change in a user's emotion by:
establishing a baseline of emotional data associated with the user by continually collecting data associated with the user;
identifying a deviation in the data by determining that a point of the collected data meets or exceeds a predetermined threshold of emotion; and
verifying the identified deviation by determining a difference between the identified deviation and the established baseline for collected data.

9. A computer program product comprising:
one or more computer readable storage media and program instructions stored on the one or more computer readable storage media, the program instructions comprising:
program instructions to collect an input from a user by transmitting instructions to at least one sensor device in a plurality of senor devices, wherein the input comprises information associated with the user;
program instructions to dynamically classify a volatile chemical by analyzing at least one result within a plurality of results for a chemical identification based on a collected input;
program instructions to determine a status of the user based on an analysis of an environment of the user and a dynamic classification of the volatile chemical, wherein the status is a condition, mood, or emotion of the user; and
program instructions to generate an adaptative model that assesses a determined status, the dynamic classification of the volatile chemical, and the collected input into a user interface within a computing device.

10. The computer program product of claim 9, wherein the program instructions to dynamically classify a volatile chemical by analyzing at least one result within a plurality of results for a chemical identification based on a collected input comprise:
program instructions to determine that a collected input matches a respective known sample in a database of known samples;
program instructions to calculate a chemical density for each collected input that matches the respective known sample based on chemical identification markers associated with a density of the respective known samples in the database of known samples;
program instructions to determine a threshold percentage of the collected input within an environment of the user by comparing the calculated chemical density to an estimated chemical density of the respective known samples; and
program instructions to verify the determined threshold percentage of the identified collected input.

11. The computer program product of claim 9, wherein the program instructions to determine a status of the user based on an analysis of an environment of the user and a dynamic classification of the volatile chemical comprise:
program instructions to examine the dynamically classified information;
program instructions to extract commonalities within the examined classified information;
program instructions to predict an ailment by performing an assessment of an extracted commonalty of the examined classification information associated with a plurality of contextual factors that indicate a presence of an ailment; and
program instructions to continually observe behavior of the user by transmitting instructions to the at least one sensor device in a plurality of sensors devices.

12. The computer program product of claim 9, wherein the program instructions stored on the one or more computer readable storage media further comprise:
program instructions to automatically update the generated adaptive model by:
program instructions to transmit data that is specific to the user to the generated adaptive model;
program instructions to dynamically prioritize each contextual factor within a compiled data based on the user specific data within the generated adaptive model; and
program instructions to automatically update the generated adaptive model based on a quantitive value of the contextual factors.

13. The computer program product of claim 12, wherein the program instructions to dynamically prioritize each contextual factor within a compiled data based on the user specific data within the generated adaptive model comprise:
program instructions to assign quantitative values to each contextual factor in a plurality of the contextual factors associated with the user specific data, wherein each contextual factor is associated with a respective user;
program instructions to calculate an overall score respective of the contextual factors associated with the user specific data, wherein the overall score is a summation of the assigned quantitative values of the contextual factors; and
program instructions to arrange the respective overall score of the contextual factors associated with the user specific data in a sequential manner having overall scores having a greater value assigned a higher order than the overall scores having a lesser value using machine learning algorithms.

14. The computer program product of claim 12, wherein the program instructions to automatically update the generated adaptive model based on a quantitive value of the contextual factors comprise:
program instructions to verify the prioritized order of the user specific input based on extracted commonalities within the user specific input;
program instructions to identify changes in the generated adaptive model by determining a difference between a recalculated overall score and an original calculated overall score; and
program instructions to modify the generated adaptive model to reflect the identified changes using a reinforced learning algorithm.

15. The computer program product of claim 9, wherein the program instructions to determine a status of the user comprise:
program instructions to create an environment around the user using IoT devices in conjunction with sensor devices;
program instructions to assign a positive value to a predetermined action or progression within a user's physiological ailment and assigning a negative value to a predetermined action or progression within a user's physiological ailment;
program instructions to calculate an overall score associated with the user's physiological ailment by aggregating the assigned values of the predetermined actions or progressions within the user's physiological ailment; and
in response to program instructions to receive additional information associated with a user's environment and physiological ailment, program instructions to dynamically modify the calculated overall score.

16. The computer program product of claim 9, wherein the program instructions stored on the one or more computer-readable storage media further comprise:
program instructions to assess a change in a user's emotion by:
program instructions to establish a baseline of emotional data associated with the user by continually collecting data associated with the user;
program instructions to identify a deviation in the data by determining that a point of the collected data meets or exceeds a predetermined threshold of emotion; and
program instructions to verify the identified deviation by determining a difference between the identified deviation and the established baseline for collected data.

17. A computer system comprising:
one or more computer processors;
one or more computer readable storage media; and
program instructions stored on the one or more computer readable storage media for execution by at least one of the one or more processors, the program instructions comprising:
program instructions to collect an input from a user by transmitting instructions to at least one sensor device in a plurality of senor devices, wherein the input comprises information associated with the user;
program instructions to dynamically classify a volatile chemical by analyzing at least one result within a plurality of results for a chemical identification based on a collected input;
program instructions to determine a status of the user based on an analysis of an environment of the user and a dynamic classification of the volatile chemical, wherein the status is a condition, mood, or emotion of the user; and
program instructions to generate an adaptative model that assesses a determined status, the dynamic classification of the volatile chemical, and the collected input into a user interface within a computing device.

18. The computer system of claim 17, wherein program instructions to dynamically classify a volatile chemical by analyzing at least one result within a plurality of results for a chemical identification based on a collected input comprise:
program instructions to determine that a collected input matches a respective known sample in a database of known samples;
program instructions to calculate a chemical density for each collected input that matches the respective known sample based on chemical identification markers associated with a density of the respective known samples in the database of known samples;
program instructions to determine a threshold percentage of the collected input within an environment of the user by comparing the calculated chemical density to an estimated chemical density of the respective known samples; and
program instructions to verify the determined threshold percentage of the identified collected input.

19. The computer system of claim 17, wherein program instructions to determine a status of the user based on an analysis of an environment of the user and a dynamic classification of the volatile chemical comprise:
program instructions to examine the dynamically classified information;
program instructions to extract commonalities within the examined classified information;
program instructions to predict an ailment by performing an assessment of an extracted commonalty of the examined classification information associated with a plurality of contextual factors that indicate a presence of an ailment; and
program instructions to continually observe behavior of the user by transmitting instructions to the at least one sensor device in a plurality of sensors devices.

20. The computer system of claim 17, wherein the program instructions stored on the one or more computer-readable storage media further comprise:
program instructions to automatically update the generated adaptive model by:
program instructions to transmit data that is specific to the user to the generated adaptive model;
program instructions to dynamically prioritize each contextual factor within a compiled data based on the user specific data within the generated adaptive model; and
program instructions to automatically update the generated adaptive model based on a quantitive value of the contextual factors.
